(19) United States
(12) Patent Application Publication
(10) Pub. No.: US 2025/0018199 A1
(43) Pub. Date: Jan. 16, 2025

(54) SYSTEMS AND METHODS FOR DETECTING SIGNAL EXTREMUM

(71) Applicant: Boston Scientific Neuromodulation Corporation, Valencia, CA (US)

(72) Inventor: Philip Leonard Weiss, Sherman Oaks, CA (US)

(21) Appl. No.: 18/767,639

(22) Filed: Jul. 9, 2024

Related U.S. Application Data
(60) Provisional application No. 63/526,950, filed on Jul. 14, 2023.

(51) Int. Cl. A61N 1/372; A61N 1/36

(52) U.S. Cl. CPC A61N 1/37241; A61N 1/36125

(57) ABSTRACT

A system may include data acquisition circuitry, local extremum detection circuitry, difference monitoring circuitry, comparator circuitry and extremum data recorder circuitry. The data acquisition circuitry may be configured to access a series of data samples from a sensor signal. The local extremum detection circuitry may be configured to find a local extremum in the series of data samples. The difference monitoring circuitry may be configured to determine a difference between the local extremum and the series of data samples subsequent to and receding from the local extremum toward the first direction. The comparator circuitry may be configured to compare the difference to a predefined threshold. The extremum data recorder circuitry may be configured to write extremum data into storage when the difference exceeds the predefined threshold.

This application claims the benefit of U.S. Provisional Application No. 63/526,950 filed on Jul. 14, 2023, which is hereby incorporated by reference in its entirety.

### TECHNICAL FIELD

This document relates generally to medical systems, and more particularly, but not by way of limitation, to systems, devices, and methods for detecting extrema in signals such as local maxima and/or minima in sensed physiological signals.

### BACKGROUND

Signals may be analyzed to evaluate and characterize systems. Signal features such as local extrema (e.g., minima and maxima) may be used to derive information about the system. By way of example and not limitation, physiological signals may be used to evaluate a patient. A patient state, for example, may be sensed by detecting one or more sensed signals. The determined patient state may be used to trigger an initiation of a therapy, a suspension of a therapy, or a change in the therapy. The detected signal may closely correspond to the intended effect of therapy such that information from the detected window may be used to provide closed-loop control of the stimulation or to evaluate an efficacy of different stimulation programs. By way of example and not limitation, neurostimulation has been proposed as a therapy for a number of conditions. Often, neurostimulation and neuromodulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES). Neurostimulation systems may deliver the therapy based on sensed physiological signals.

Sensed physiological signals, such as but not limited to, may have a complex morphology. Furthermore, noise may add to the complexity of the signal. Some examples of physiological signals with complex morphologies include local field potentials and evoked compound action potentials (ECAPs) and evoked resonant neural activity (ERNA). Other examples of sensed physiological signals may include cardiac activity (e.g., electrocardiogra (ECG)), muscle activity (e.g., electromyography (EMG)), brain activity (e.g., electroencephalography (EEG)), electroneuronography (ENOG)) and galvanic skin responses (GSR). Other examples of sensed physiological signals include impedance (e.g., respiration sensors/transthoracic impedance) or movement (e.g., movement detected using an accelerometer or camera). The movement may be for the whole person or for a portion of the patient such limb movement, head movement, or eye movement.

Extrema in the signals, including peaks (e.g., also referred to as local maximums) and troughs (also referred to as a local minimums), can contain useful information. However, there are challenges associated with accurately detecting extrema in signals. For example, the detected extrema should correspond to a number of extrema expected to be found within a size of a sampling window. Also, noise may be present in the signals. Thus, some fluctuations in the signal should be ignored and should not be classified as a local extremum.

### SUMMARY

By way of example and not limitation, various embodiments provided herein use at least one threshold for ignoring some signal fluctuations and classifying other signal fluctuations as local extrema.

An example (e.g., Example 1) of a system may include data acquisition circuitry, local extremum detection circuitry, difference monitoring circuitry, comparator circuitry and extremum data recorder circuitry. The data acquisition circuitry may be configured to access a series of data samples from a sensor signal. The local extremum detection circuitry may be configured to find a local extremum in the series of data samples. The local extremum is a data sample where at least one data sample immediately preceding the local extremum approaches from a first direction and at least one data sample immediately succeeding the local extremum recedes toward the first direction. The difference monitoring circuitry may be configured to determine a difference between the local extremum and the series of data samples subsequent to and receding from the local extremum toward the first direction. The comparator circuitry may be configured to compare the difference to a predefined threshold. The extremum data recorder circuitry may be configured to write extremum data into storage when the difference exceeds the predefined threshold.

In Example 2, the subject matter of Example 1 may optionally be configured to further include a sensor configured to sense a biological parameter from a patient and provide the sensor signal, and a sensor signal sampler configured to sample the sensor signal to provide the series of data samples.

In Example 3, the subject matter of Example 2 may optionally be configured such that the data acquisition circuitry is configured to access a stream of data samples.

In Example 4, the subject matter of any one or more of Examples 2-3 may optionally be configured to further include a stimulator configured to deliver electrical stimulation. The data acquisition circuitry may be configured to use the sensor to sense a response to the electrical stimulation.

In Example 5, the subject matter of Example 4 may optionally be configured such that the data acquisition circuitry is configured to access sample data within a window after a stimulation pulse and timed to avoid a stimulation artifact and to capture an evoked potential from the delivered electrical stimulation.

In Example 6, the subject matter of any one or more of Examples 2-5 may optionally be configured such that the sensor includes an evoked compound action potential (ECAP) sensor, a local field potential (LFP) sensor, an evoked resonant neural activity (ERNA) sensor, or a cardiac activity sensor.

In Example 7, the subject matter of any one or more of Examples 1-6 may optionally be configured such that the local extremum detection circuitry is configured to find the local extremum by determining a potential extremum using a datapoint in the series of sample data, and updating the potential extremum when the difference did not reach the predefined threshold and a subsequent datapoint in the series of sample data is more extreme. The potential extremum may be referred to as a candidate extremum, which is classified as a local extremum when the difference reaches the threshold.

In Example 8, the subject matter of any one or more of Examples 1-7 may optionally be configured such that, when the local extremum is a local maxima, the predefined threshold is a predefined local maxima threshold, the series of sample data points immediately subsequent to the local maxima is less than the local maxima, and local maxima data is written into the storage when the series of sample data points is less than the local maximal by at least the predefined local maxima threshold.

In Example 9, the subject matter of any one or more of Examples 1-8 may optionally be configured such that, when the local extremum is a local minima, the predefined threshold is a predefined local minima threshold, the series of sample data points immediately subsequent to the local minima is greater than the local minima, and local minima data is written into the storage when the series of sample data points is more than the local minima by at the predefined local minima threshold.

In Example 10, the subject matter of any one or more of Examples 1-9 may optionally be configured such that the storage includes First In First Out (FIFO) storage, and the extremum data recorder circuitry is configured to write extremum data into storage by writing extremum data into the FIFO and then from the FIFO into a persistent memory.

In Example 11, the subject matter of any one or more of Examples 1-10 may optionally be configured such that the local extremum detection circuitry is further configured to perform recursive averaging to reduce noise in finding the local extremum.

In Example 12, the subject matter of any one or more of Examples 1-11 may optionally be configured to further include a medical device programmer with a user-interface configured to receive the predefined threshold.

In Example 13, the subject matter of any one or more of Examples 1-12 may optionally be configured such that the extremum data written into the storage includes at least one of: a value for the extremum data; or a length or an amplitude of a chord between successive local extremums.

In Example 14, the subject matter of any one or more of Examples 1-13 may optionally be configured such that the comparator circuitry is further configured to compare the local extremum to an expected extremum to provide a confidence indicator for the extremum date written into the storage.

In Example 15, the subject matter of any one or more of Examples 1-14 may optionally be configured such that the local extremum detection circuitry, the difference monitoring circuitry, the comparator circuitry and the extremum data recorder circuitry are configured to operate on each of a plurality of local extremums in the series of sample data points.

Example 16 includes subject matter (such as a method, means for performing acts, machine readable medium including instructions that when performed by a machine cause the machine to perform acts, or an apparatus to perform). The subject matter may include accessing a series of data samples from a sensor signal, finding a local extremum in the series of data samples where at least one data sample immediately preceding the local extremum approaches the local extremum from a first direction and at least one data sample immediately succeeding the local extremum recedes from the local extremum toward the first direction, determining a difference between the local extremum and the series of data samples subsequent to and receding from the local extremum toward the first direction, comparing the difference to a predefined threshold, and writing extremum data into storage when the difference exceeds the predefined threshold.

In Example 17, the subject matter of Example 16 may optionally be configured to further include using a sensor to sense a biological parameter from a patient and provide the sensor signal, and sampling the sensor signal to provide the series of data samples.

In Example 18, the subject matter of any one or more of Examples 16-17 may optionally be configured such that the series of data samples is accessed from a stream of data samples.

In Example 19, the subject matter of any one or more of Examples 16-18 may optionally be configured to further include delivering electrical stimulation to the patient, and sensing a response to the electrical stimulation by sensing the biological parameter.

In Example 20, the subject matter of Example 19 may optionally be configured such that the accessing the sequence of sample data includes accessing sample data within a window after a stimulation pulse and timed to avoid a stimulation artifact and to capture an evoked potential from the delivered electrical stimulation.

In Example 21, the subject matter of any one or more of Examples 17-20 may optionally be configured such that the sensor includes an evoked compound action potential (ECAP) sensor, a local field potential (LFP) sensor, an evoked resonant neural activity (ERNA) sensor, or a cardiac activity sensor.

In Example 22, the subject matter of any one or more of Examples 16-21 may optionally be configured such that the finding the local extremum includes determining a potential extremum using a datapoint in the series of sample data, and updating the potential extremum when the difference did not reach the predefined threshold and a subsequent datapoint in the series of sample data is more extreme.

In Example 23, the subject matter of any one or more of Examples 16-22 may optionally be configured such that when the local extremum is a local maxima, the predefined threshold is a predefined local maxima threshold, the series of sample data points immediately subsequent to the local maxima is less than the local maxima, and local maxima data is written into the storage when the series of sample data points is less than the local maximal by at least the predefined local maxima threshold.

In Example 24, the subject matter of any one or more of Examples 16-23 may optionally be configured such that when the local extremum is a local minima, the predefined threshold is a predefined local minima threshold, the series of sample data points immediately subsequent to the local minima is greater than the local minima, and local minima data is written into the storage when the series of sample data points is more than the local minima by at the predefined local minima threshold.

In Example 25, the subject matter of any one or more of Examples 16-24 may optionally be configured such that the storage includes First In First Out (FIFO) storage, and the writing extremum data into storage includes writing extremum data into the FIFO and then from the FIFO into a persistent memory.

In Example 26, the subject matter of any one or more of Examples 16-25 may optionally be configured to further include performing recursive averaging to reduce noise in finding the local extremum.

In Example 27, the subject matter of any one or more of Examples 16-26 may optionally be configured to further include receiving user-programming via a medical device programmer to set the predefined threshold.

In Example 28, the subject matter of any one or more of Examples 16-27 may optionally be configured such that the extremum data written into the storage includes at least one of: a value for the extremum data; or a length or an amplitude of a chord between successive local extremums.

In Example 29, the subject matter of any one or more of Examples 16-28 may optionally be configured to further include comparing the local extremum to an expected extremum to provide a confidence indicator for the extremum date written into the storage.

In Example 30, the subject matter of any one or more of Examples 16-29 may optionally be configured such that the finding, the determining, the comparing, and the writing is performed for each of a plurality of local extremums in the series of sample data points.

Example 31 includes subject matter (such as a non-transitory machine-readable medium including instructions, which when executed by a machine, cause the machine to perform a method for identifying effective placement of at least one lead having a plurality of electrodes) The method performed using the machine may include accessing a series of data samples from a sensor signal, finding a local extremum in the series of data samples where at least one data sample immediately preceding the local extremum approaches the local extremum from a first direction and at least one data sample immediately succeeding the local extremum recedes from the local extremum toward the first direction, determining a difference between the local extremum and the series of data samples subsequent to and receding from the local extremum toward the first direction, comparing the difference to a predefined threshold, and writing extremum data into storage when the difference exceeds the predefined threshold. In additional examples, the subject matter of Examples 17-30 may be performed by the machine.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neuromodulation system.

FIG. 2 illustrates an embodiment of a modulation device, such as may be implemented in the neuromodulation system of FIG. 1.

FIG. 3 illustrates an embodiment of a programming system such as a programming device, which may be implemented as the programming device in the neuromodulation system of FIG. 1.

FIG. 4 illustrates, by way of example, an implantable neuromodulation system and portions of an environment in which system may be used.

FIG. 5 illustrates, by way of example, an embodiment of a SCS system, which also may be referred to as a Spinal Cord Modulation (SCM) system.

FIG. 6 illustrates, by way of example and not limitation, an example of a sensed signal and the application of a threshold to which fluctuations are ignored and which fluctuations are to be considered to be an extremum (e.g., a local minimum or local maximum).

FIG. 7 illustrates, by way of example and not limitation, data that may be stored for extrema.

FIG. 8 illustrates, by way of example and not limitation, a method for evaluating sample data for a sensed signal to find local extrema.

FIG. 9 illustrates, by way of example and not limitation, a more specific example for evaluating sample data for a signal to find both local minima and local maxima.

FIG. 10 illustrates, by way of example and not limitation, a state diagram for identifying and capturing local minima and local maxima data in a sensed signal.

FIG. 11 illustrates, by way of example and not limitation, a system for evaluating data samples from a sensed signal to identify and capture local extrema data.

FIG. 12 illustrates, by way of example and not limitation, a system for capturing extrema data from data samples.

FIGS. 13A and 13B illustrate, by way of example and not limitation, examples of recursive averaging of extrema data.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides systems, devices, and methods for automatically finding local minima and/or local maxima in a signal using at least one predefined threshold. The threshold(s) may be used to classify fluctuations within the signal in order to determine if a given fluctuation is something to be ignored as small or unimportant features such as noise or if the given fluctuation is something that should be considered as an extremum (e.g., a local maximum or local minimum) within the signal. Signal data corresponding to the extremum may be stored in a FIFO and, as the FIFO fills, the signal data may be stored into other memory (e.g., nonvolatile such as persistent memory) for later analysis or immediate use. Some embodiments may use recursive averaging of sensed data to provide additional resistance to noise. A benefit of the present subject matter includes, but is not limited to, a more power efficient process for detecting peaks and troughs in a signal compared to a processor that processes each of the samples in the sample data.

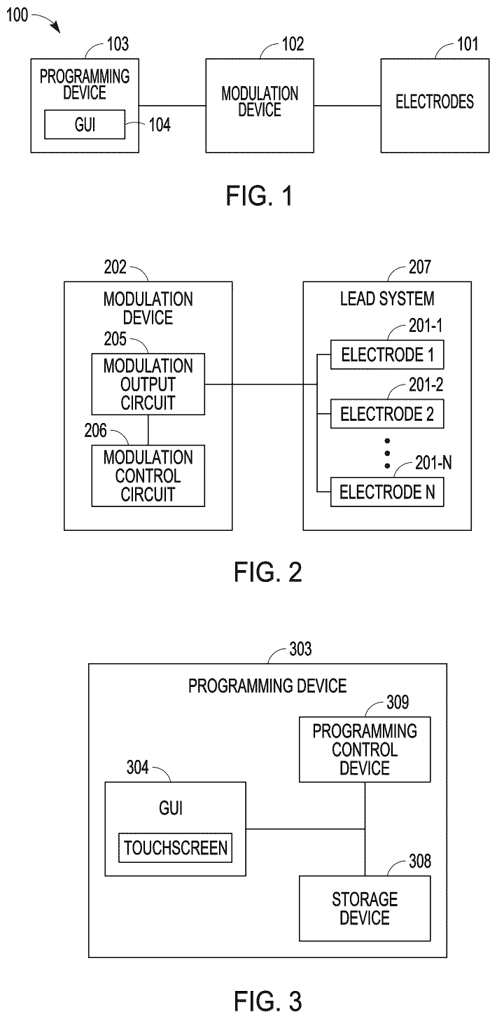

FIG. 1 illustrates, by way of example and not limitation, an embodiment of a neuromodulation system. The illustrated system 100 includes electrode contacts 101, which also may be simply referred to as electrodes, a modulation device 102, and a programming system such as a programming device 103. The programming system may include multiple devices. The electrode contacts 101 are configured to be placed on or near one or more neural targets in a patient. The modulation device 102 is configured to be electrically connected to electrode contacts 101 and deliver neuromodulation energy, such as in the form of electrical pulses, to the one or more neural targets though electrode contacts 101. The delivery of the neuromodulation is controlled by using a plurality of modulation parameters. The modulation parameters may specify the electrical waveform (e.g., pulses or pulse patterns or other waveform shapes) and a selection of electrode contacts through which the electrical waveform is delivered. In various embodiments, at least some parameters of the plurality of modulation parameters are programmable by a user, such as a physician or other caregiver. The programming device 103 provides the user with accessibility to the user-programmable parameters. In various embodiments, the programming device 103 is configured to be communicatively coupled to modulation device via a wired or wireless link. In various embodiments, the programming device 103 includes a graphical user interface (GUI) 104 that allows the user to set and/or adjust values of the user-programmable modulation parameters.

Some embodiments of the programming device and/or modulation device may access sensed data samples. The programming and/or control of the therapy may be based on the sensed data samples (e.g., extrema within the sensed data sample). The sensed data samples may be based on an electrical signal sensed using at least some of the electrodes 101.

FIG. 2 illustrates an embodiment of a modulation device 202, such as may be implemented in the neuromodulation system 100 of FIG. 1. The modulation device 202 may also be referred to as a neurostimulator. Various embodiments of the neurostimulator may be used to deliver different types of neural therapy such as, but not limited to, SCS, DBS, PNS or FES therapy. The illustrated embodiment of the modulation device 202 includes a modulation output circuit 205 and a modulation control circuit 206. Those of ordinary skill in the art will understand that the neuromodulation system may include additional components such as sensing circuitry for patient monitoring and/or feedback control of the therapy, telemetry circuitry and power. Some embodiments of the modulation device 202 may include storage (e.g., FIFO and/or other storage) for storing sensed data samples and/or local extrema data for a signal. The modulation output circuit 205 produces and delivers the neuromodulation. Neuromodulation pulses are provided herein as an example. However, the present subject matter is not limited to pulses, but may include other electrical waveforms (e.g., waveforms with different waveform shapes, and waveforms with various pulse patterns). The modulation control circuit 206 controls the delivery of the neuromodulation pulses using the plurality of modulation parameters. The lead system 207 includes one or more leads each configured to be electrically connected to modulation device 202 and a plurality of electrode contacts 201-1 to 201-N distributed in an electrode contact arrangement using the one or more leads. Each lead may have an electrode contact array consisting of two or more electrode contacts, which also may be referred to as electrodes. Multiple leads may provide multiple electrode contact arrays to provide the electrode contact arrangement. Each electrode contact is a single electrically conductive contact providing for an electrical interface between modulation output circuit 205 and tissue of the patient, where N≥2. The neuromodulation pulses are each delivered from the modulation output circuit 205 through a set of electrode contacts selected from the electrode contacts 201-1 to 201-N. The number of leads and the number of electrode contacts on each lead may depend on, for example, the distribution of target(s) of the neuromodulation and the need for controlling the distribution of electric field at each target. In one embodiment, by way of example and not limitation, the lead system includes two leads each having eight electrode contacts. Some embodiments may use a lead system that includes a paddle lead.

The actual number and shape of leads and electrode contacts may vary for the intended application. An implantable waveform generator may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode contact (e.g., case electrode). The waveform generator may include electronic components, such as a controller/processor (e.g., a microcontroller), memory, a battery, telemetry circuitry, monitoring circuitry, modulation output circuitry, and other suitable components known to those skilled in the art. The microcontroller executes a suitable program stored in memory, for directing and controlling the neuromodulation performed by the waveform generator. Electrical modulation energy is provided to the electrode contacts in accordance with a set of modulation parameters programmed into the pulse generator. By way of example but not limitation, the electrical modulation energy may be in the form of a pulsed electrical waveform. Such modulation parameters may comprise electrode contact combinations, which define the electrode contacts that are activated as anodes (positive), cathodes (negative), and turned off (zero), percentage of modulation energy assigned to each electrode contact (fractionalized electrode contact configurations), and electrical pulse parameters, which define the pulse amplitude (measured in milliamps or volts depending on whether the pulse generator supplies constant current or constant voltage to the electrode contact array), pulse width (measured in microseconds), pulse rate (measured in pulses per second), and burst rate (measured as the modulation on duration X and modulation off duration Y). Electrode contacts that are selected to transmit or receive electrical energy are referred to herein as “activated,” while electrode contacts that are not selected to transmit or receive electrical energy are referred to herein as “non-activated.”

Electrical modulation occurs between or among a plurality of activated electrode contacts, one of which may be the case of the waveform generator. The system may be capable of transmitting modulation energy to the tissue in a monopolar or multipolar (e.g., bipolar, tripolar, etc.) fashion. Monopolar modulation occurs when a selected one of the lead electrode contacts is activated along with the case of the waveform generator, so that modulation energy is transmitted between the selected electrode contact and case. Any of the electrode contacts E1-E16 and the case electrode contact may be assigned to up to k possible groups or timing “channels.” In one embodiment, k may equal four. The timing channel identifies which electrode contacts are selected to synchronously source or sink current to create an electric field in the tissue to be stimulated. Amplitudes and polarities of electrode contacts on a channel may vary. In particular, the electrode contacts can be selected to be positive (anode, sourcing current), negative (cathode, sinking current), or off (no current) polarity in any of the k timing channels. The waveform generator may be operated in a mode to deliver electrical modulation energy that is therapeutically effective and causes the patient to perceive delivery of the energy (e.g., therapeutically effective to relieve pain with perceived paresthesia), and may be operated in a sub-perception mode to deliver electrical modulation energy that is therapeutically effective and does not cause the patient to perceive delivery of the energy (e.g., therapeutically effective to relieve pain without perceived paresthesia). The waveform generator may also be configured to deliver waveforms or pulses that are not therapeutically effective but are useful for intermittent charge balancing.

The waveform generator may be configured to individually control the magnitude of electrical current flowing through each of the electrode contacts. For example, a current generator may be configured to selectively generate individual current-regulated amplitudes from independent current sources for each electrode contact. In some embodiments, the pulse generator may have voltage regulated outputs. While individually programmable electrode contact amplitudes are desirable to achieve fine control, a single output source switched across electrode contacts may also be used, although with less fine control in programming. Neuromodulators may be designed with mixed current and voltage regulated devices.

The neuromodulation system may be configured to modulate spinal target tissue or other neural tissue. The configuration of electrode contacts used to deliver electrical pulses to the targeted tissue constitutes an electrode contact configuration, with the electrode contacts capable of being selectively programmed to act as anodes (positive), cathodes (negative), or left off (zero). In other words, an electrode contact configuration represents the polarity being positive, negative, or zero. An electrical waveform may be controlled or varied for delivery using electrode contact configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode contact configuration, along with the electrical pulse parameters, can be referred to as a “modulation parameter set.” Each set of modulation parameters, including fractionalized current distribution to the electrode contacts (as percentage cathodic current, percentage anodic current, or off), may be stored and combined into a modulation program that can then be used to modulate multiple regions within the patient.

The number of electrode contacts available combined with the ability to generate a variety of complex electrical waveforms (e.g., pulses), presents a huge selection of modulation parameter sets to the clinician or patient. For example, if the neuromodulation system to be programmed has sixteen electrode contacts, millions of modulation parameter sets may be available for programming into the neuromodulation system. Furthermore, for example SCS systems may have thirty-two electrode contacts which exponentially increases the number of modulation parameters sets available for programming. To facilitate such selection, the clinician generally programs the modulation parameters sets through a computerized programming system to allow the optimum modulation parameters to be determined based on patient feedback, sensor feedback or other means and to subsequently program the desired modulation parameter sets.

FIG. 3 illustrates an embodiment of a programming system such as a programming device 303, which may be implemented as the programming device 103 in the neuromodulation system of FIG. 1. The programming device 303 includes a storage device 308, a programming control circuit 309, and a graphical user interface (GUI) 304. The programming control circuit 309 generates the plurality of modulation parameters that control the delivery of the neuromodulation pulses according to the pattern of the neuromodulation pulses. In various embodiments, the GUI 304 includes any type of presentation device, such as interactive or non-interactive screens, and any type of user input devices that allow the user to program the modulation parameters, such as touchscreen, keyboard, keypad, touchpad, trackball, joystick, and mouse. The storage device 308 may store, among other things, modulation parameters to be programmed into the modulation device. Some embodiments may store in the storage device 308 sensed data samples and/or local extrema data for a signal. The programming device 303 may transmit the plurality of modulation parameters to the modulation device. In some embodiments, the programming device 303 may transmit power to the modulation device. The programming control circuit 309 may generate the plurality of modulation parameters. In various embodiments, the programming control circuit 309 may check values of the plurality of modulation parameters against safety rules to limit these values within constraints of the safety rules.

In various embodiments, circuits of neuromodulation, including its various embodiments discussed in this document, may be implemented using a combination of hardware, software and firmware. For example, the GUI circuit, modulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

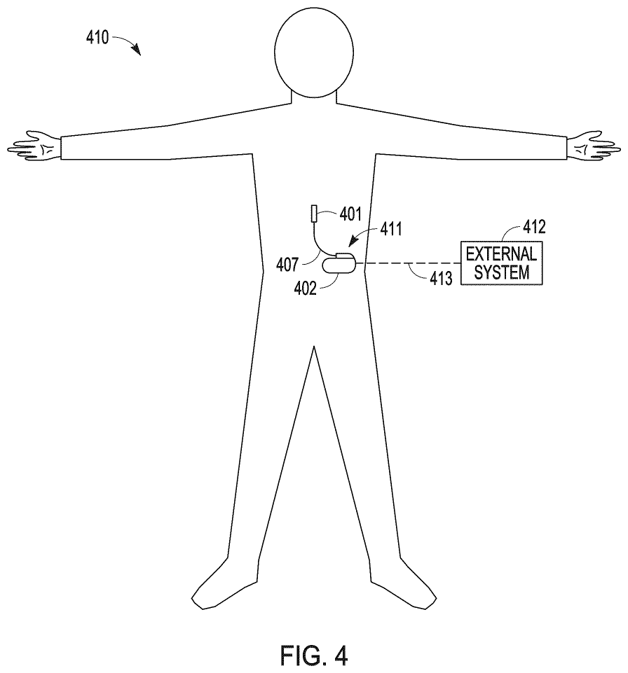

FIG. 4 illustrates, by way of example, an implantable neuromodulation system and portions of an environment in which system may be used. The system is illustrated for implantation near the spinal cord. However, neuromodulation system may be configured to modulate other neural targets including, but not limited to, SBS, PNS or FES targets. The system 410 includes an implantable system 411, an external system 412, and a telemetry link 413 providing for wireless communication between implantable system 411 and external system 412. The implantable system is illustrated as being implanted in the patient's body. The implantable system 411 includes an implantable modulation device (also referred to as an implantable pulse generator, or IPG) 402, a lead system 407, and electrode contacts 401. The lead system 407 includes one or more leads each configured to be electrically connected to the modulation device 402 and a plurality of electrode contacts 401 distributed in the one or more leads. In various embodiments, the external system 412 includes one or more external (non-implantable) devices each allowing a user (e.g., a clinician or other caregiver and/or the patient) to communicate with the implantable system 411. In some embodiments, the external system 412 includes a programming device intended for a clinician or other caregiver to initialize and adjust settings for the implantable system 411 and a remote control device intended for use by the patient. For example, the remote control device may allow the patient to turn a therapy on and off and/or adjust certain patient-programmable parameters of the plurality of modulation parameters. The external system 412 may include personal devices such as phones and tablets. The external system may include other processing and/or storage device(s). Some external device examples include a device programmer, a remote control, a phone or tablet, a local system (e.g., local computer, network of computers, network attached storage (NAS), or portable data storage such as a flash drive), or a remote system (e.g., cloud-based systems).

The neuromodulation lead(s) of the lead system 407 may be placed adjacent, i.e., resting near, or upon the dura, adjacent to the spinal cord area to be stimulated. For example, the neuromodulation lead(s) may be implanted along a longitudinal axis of the spinal cord of the patient. Due to the lack of space near the location where the neuromodulation lead(s) exit the spinal column, the implantable modulation device 402 may be implanted in a surgically-made pocket either in the abdomen or above the buttocks, or may be implanted in other locations of the patient's body. The lead extension(s) may be used to facilitate the implantation of the implantable modulation device 402 away from the exit point of the neuromodulation lead(s).

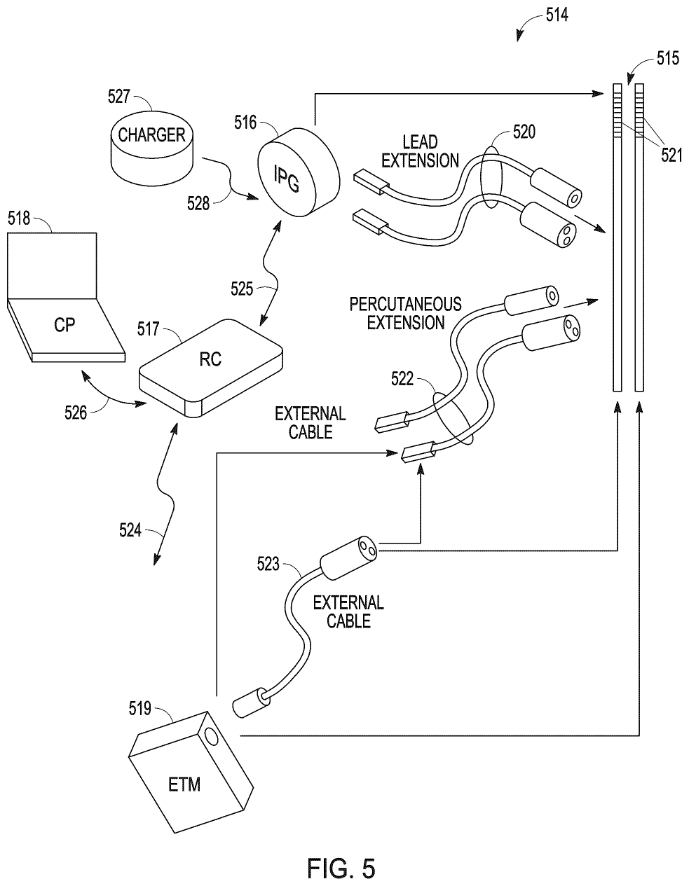

FIG. 5 illustrates, by way of example, an embodiment of a SCS system, which also may be referred to as a Spinal Cord Modulation (SCM) system. A similar system, with DBS lead(s), may be used to provide a DBS system. The SCS system 514 may generally include one or more (illustrated as two) of implantable neuromodulation leads 515, an electrical waveform generator 516 such as an implantable pulse generator, an external remote controller (RC) 517, a clinician's programmer (CP) 518, and an external trial modulator (ETM) 519. IPGs are used herein as an example of the electrical waveform generator. However, it is expressly noted that the waveform generator may be configured to deliver regular, repeating patterns of pulses or in complex patterns that appear to be irregular patterns of pulses where pulses have differing amplitudes, pulse widths, pulse intervals, and bursts with differing number of pulses. It is also expressly noted that the waveform generator may be configured to deliver electrical waveforms other than pulses. The waveform generator 516 may include pulse generation circuitry that delivers electrical modulation energy in the form of a pulsed electrical waveform (i.e., a temporal series of electrical pulses) to the electrode contacts in accordance with a set of modulation parameters. The electrical waveform may include first phases of a first polarity and second phases of a second polarity opposite the first polarity. Neural tissue may be therapeutically stimulated using both the first phases and the second phases of the electrical waveform. The electrical waveform includes a plurality of interphase intervals, each of the plurality of interphase intervals separating individual ones of the first phases and individual ones of the second phases. The second phases may be used to reduce built up charge from the at least one electrode contact caused by the first phases and the first phases may be used to reduce built up charge from the at least one electrode contact caused by the second phases. The waveform generator 516 may be physically connected via one or more percutaneous lead extensions 520 to the neuromodulation leads 515, which carry a plurality of electrode contacts 521. As illustrated, the neuromodulation leads 515 may be percutaneous leads with the electrode contacts arranged in-line along the neuromodulation leads. Any suitable number of neuromodulation leads can be provided, including only one, as long as the number of electrode contacts is greater than two (including the waveform generator case function as a case electrode contact) to allow for lateral steering of the current. Alternatively, a surgical paddle lead can be used in place of one or more of the percutaneous leads.

The ETM 519 may also be physically connected via the percutaneous lead extensions 522 and external cable 523 to the neuromodulation leads 515. The ETM 519 may have similar waveform generation circuitry as the waveform generator 516 to deliver electrical modulation energy to the electrode contacts accordance with a set of modulation parameters. The ETM 519 is a non-implantable device that is used on a trial basis after the neuromodulation leads 515 have been implanted and prior to implantation of the waveform generator 516, to test the responsiveness of the modulation that is to be provided. Functions described herein with respect to the waveform generator 516 can likewise be performed with respect to the ETM 519.

The RC 517 may be used to telemetrically control the ETM 519 via a bi-directional RF communications link 524. The RC 517 may be used to telemetrically control the waveform generator 516 via a bi-directional RF communications link 525. Such control allows the waveform generator 516 to be turned on or off and to be programmed with different modulation parameter sets. The waveform generator 516 may also be operated to modify the programmed modulation parameters to actively control the characteristics of the electrical modulation energy output by the waveform generator 516. A clinician may use the CP 518 to program modulation parameters into the waveform generator 516 and ETM 519 in the operating room and in follow-up sessions. The waveform generator 516 may be implantable. The implantable waveform generator 516 and the ETM 519 may have similar features as discussed with respect to the modulation device 202 described with respect to FIG. 2.

The CP 518 may indirectly communicate with the waveform generator 516 or ETM 519, through the RC 517, via an IR communications link 526 or other link. The CP 518 may directly communicate with the waveform generator 516 or ETM 519 via an RF communications link or other link (not shown). The clinician detailed modulation parameters provided by the CP 518 may also be used to program the RC 517, so that the modulation parameters can be subsequently modified by operation of the RC 517 in a stand-alone mode (i.e., without the assistance of the CP 518). Various devices may function as the CP 518. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 518. Alternatively, such programming methodologies can be performed using firmware or hardware. In any event, the CP 518 may actively control the characteristics of the electrical modulation generated by the waveform generator 516 to allow the desired parameters to be determined based on patient feedback, sensor feedback or other feedback and for subsequently programming the waveform generator 516 with the desired modulation parameters. To allow the user to perform these functions, the CP 518 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant waveform generator, implant waveform generator and lead(s), replace waveform generator, replace waveform generator and leads, replace or revise leads, explant, etc.), generate a pain map of the patient, define the configuration and orientation of the leads, initiate and control the electrical modulation energy output by the neuromodulation leads, and select and program the IPG with modulation parameters in both a surgical setting and a clinical setting.

An external charger 527 may be a portable device used to transcutaneously charge the waveform generator via a wireless link such as an inductive link 528. Once the waveform generator has been programmed, and its power source has been charged by the external charger or otherwise replenished, the waveform generator may function as programmed without the RC or CP being present.

The neurostimulation may be based on sensed physiological signals. Examples of such signals may include, but are not limited to, local field potentials, evoked compound action potentials (ECAPs), evoked resonant neural activity (ERNA), cardiac activity (e.g., electrocardiogramuscle activity (e.g., electromyography (EMG)), brain activity (e.g., electroencephalography (EEG)), electroneuronography (ENOG)), galvanic skin responses (GSR), impedance, or movement (e.g., movement detected using an accelerometer or camera). For example, the signals may be used to program the desired neurostimulation parameters and/or may be used to control the timing of the therapy (e.g., when to initiate the therapy, when to suspend the therapy, and/or when and/or how to change the therapy). Extrema in the signals, including peaks (e.g., also referred to as local maximums) and troughs (also referred to as a local minimums), can contain useful information. Various embodiments of present subject matter provide systems and methods for finding a local extremum (e.g., peaks and/or troughs) in a signal (e.g., a series of data samples). The systems and methods may continuously check for local minimums and maximums within streaming data or within a window of time. A threshold for finding the extremum may be predefined. For example, the threshold may be entered or controlled by the user or may be otherwise predefined and stored in a manner accessible by the system. Some embodiments may use different thresholds for finding local maxima (peaks) and for finding local minima (troughs). As the local extrema are detected, the extrema information may be stored in a First In First Out (FIFO) storage. As the FIFO fills, the information may be stored into another memory for later analysis or for immediate use. This other memory may be within an implantable device such as a neuromodulator or may be in an external device such as a device programmer, a remote control, a phone or tablet, a local system (e.g., local computer, network of computers, network attached storage (NAS), or portable data storage such as a flash drive), or a remote system (e.g., cloud-based systems). The memory may be nonvolatile memory such as but not limited to persistent memory (a non-volatile, low-latency memory that provides a processor with fast access to data). The movement of the signal away from a candidate local extremum is compared against a threshold to determine that the candidate local extremum is classified and written as a local extremum to storage (e.g., to the FIFO). For example, a candidate local extremum has immediately preceding data sample(s) approaching the candidate local extremum from a first direction and immediately succeeding data sample(s) receding from the local extremum toward the first direction. A difference may be determined between the local extremum and the series of data samples subsequent to and receding from the local extremum toward the first direction, comparing the difference to a predefined threshold, and writing extremum data into storage when the difference exceeds the predefined threshold. The threshold may be predefined to effectively filter out small, unimportant, peaks and noise. Peak detection can work inside a window or with continuous data. As long as the data can be emptied when the FIFO is full, unlimited extrema data may be detected and written to the FIFO. Extrema data may include a value for the extremum data (e.g., peak or trough value); or a length or an amplitude of a chord between successive local extremums.

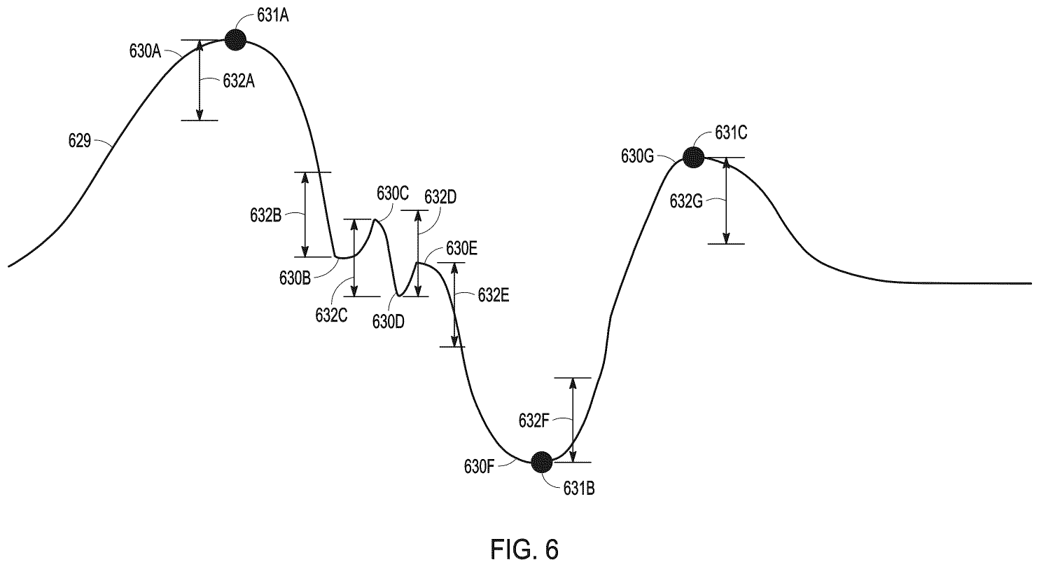

FIG. 6 illustrates, by way of example and not limitation, an example of a sensed signal and the application of a threshold to which fluctuations are ignored and which fluctuations are to be considered to be an extremum (e.g., a local minimum or local maximum). The illustrated signal 629 includes a plurality of fluctuations 630A-630G where the signal changes directions. Each of these fluctuations may be considered to be a candidate for a local extremum (e.g., a potential local extremum). However, only some of the fluctuations should be classified as local extrema (631A-631C). Embodiments of the present subject matter provide this classification using a predefined threshold. For example, the signal movement before fluctuation 630A is greater than threshold 632A and the signal movement after fluctuation 630A is greater than threshold 632A. Thus, the fluctuation 630A is classified as a local extremum (local maximum or peak) 631A. The signal movement before fluctuation 630B is more than threshold 632B but the signal movement after fluctuation 630B is less than threshold 632B such that the fluctuation 630B is not classified as a local extremum. The signal movement before 630C is less than threshold 62B and the signal movement after fluctuation 630C is less than threshold 632C such that the fluctuation is not classified as a local extremum. The signal movement before fluctuation 630D is less than threshold 632C and the signal movement after fluctuation 630D is less than threshold 632D such that the fluctuation is not classified as a local extremum. The signal movement after fluctuation 630E is greater than threshold 632E but the signal movement before fluctuation 630E is less than threshold 632D such that the fluctuation is not classified as a local extremum. The signal movement after fluctuation 630F is greater than threshold 632E and the signal movement after fluctuation 630F is greater than threshold 632F such that the fluctuation 632F is classified as a local extremum. The signal movement after fluctuation 630G is greater than threshold 632F and the signal movement after fluctuation 630G is greater than threshold 632G such that the fluctuation 632G is classified as a local extremum.

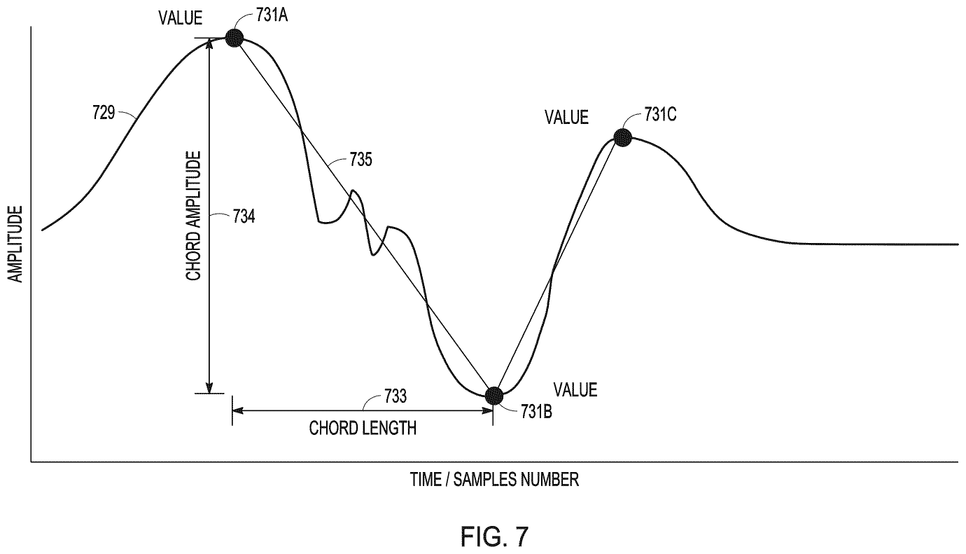

FIG. 7 illustrates, by way of example and not limitation, data that may be stored for extrema. The figure shows signal 729, similar to signal 629 in FIG. 6, with extrema 731A, 731B and 731C. The data may include values for extrema 731A, 731B and 731C. The data may also include a length 733 or an amplitude 734 of a chord (e.g., chord 735) between successive local extremums (e.g., between 731A and 731B).

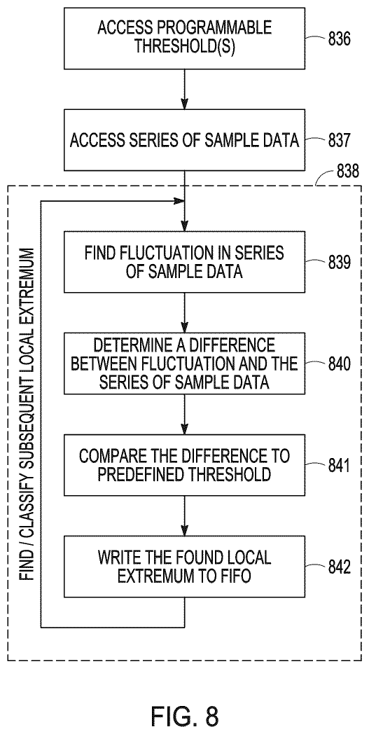

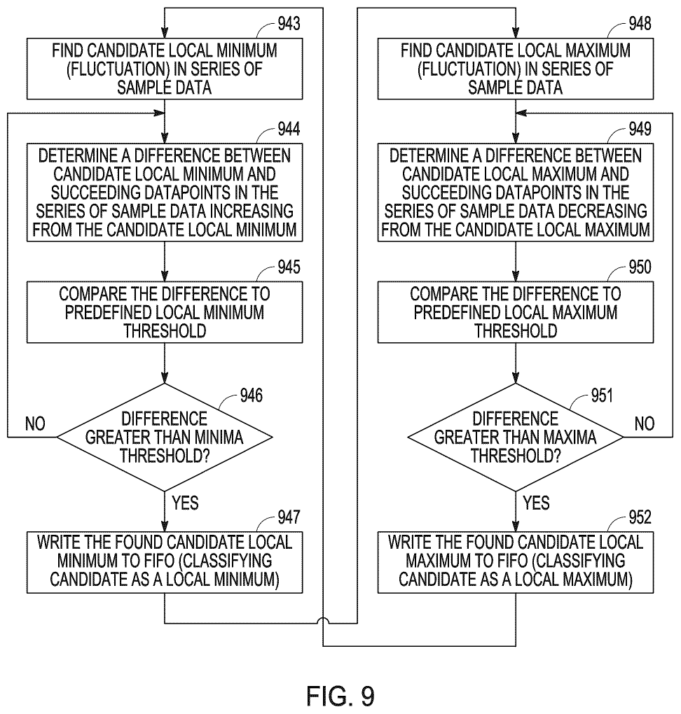

FIG. 8 illustrates, by way of example and not limitation, a method for evaluating sample data for a sensed signal to find local extrema. The illustrated method may include at 836 accessing one or more thresholds for classifying fluctuations in the signal. The threshold(s) may be programmable by a user or may be otherwise predefined (e.g., preprogrammed in the system). The method may include accessing a series of data samples from a sensor signal at 837 and finding and classifying a subsequent local extremum at 838. Finding the local extremum may include finding a fluctuation that may be a candidate local extremum in a series of sample data 839, determining a difference between the fluctuation and the series of the sample data 840, comparing the difference to the predefined threshold 841, and writing local extremum data for that fluctuation into FIFO storage 842, indicating that the fluctuation is not just a candidate but is being classified as a local extremum. At least one data sample immediately preceding the fluctuation (local extremum) approaches the local extremum from a first direction and at least one data sample immediately succeeding the local extremum recedes from the local extremum toward the first direction. The difference between the local extremum and the series of data samples subsequent to and receding from the local extremum toward the first direction, comparing the difference to a predefined threshold, and writing extremum data into storage when the difference exceeds the predefined threshold.

FIG. 9 illustrates, by way of example and not limitation, a more specific example for evaluating sample data for a signal to find both local minima and local maxima. The left side of the figure illustrates finding a local minimum and the right side of the figure illustrates finding a local maximum. Once a local minimum is found, if the signal continues to go lower without an intervening local maximum, then the current local minimum may be updated. Similarly, once a local maximum is found, if the signal continues to go higher without an intervening local minimum, then the current local maximum may be updated. At 943 a fluctuation within the signal is identified as a candidate local minimum. For example, the preceding sample(s) and succeeding sample(s) are greater than the current sample. Other means may be used to identify fluctuations in the signal. At 944 a difference is determined between the candidate local minimum and succeeding data points in the series of sample data that are increasing from the local minimum. At 945 the difference is compared to a predefined local minimum threshold. At 946 it is determined whether the distance is greater than a minima threshold. If it is not, the process may return to 944 to check subsequent sample data. If the distance is greater than a minima threshold, then at 947 the candidate local minimum is classified as a local minimum and candidate local minimum information is written to the FIFO storage. Subsequent data samples may go lower before the next local maximum is found. In such cases, the local minimum is updated based on the lower, subsequent data samples. At 948 a fluctuation within the signal is identified as a candidate local maximum. For example, the preceding sample(s) and succeeding sample(s) are less than the current sample. Other means may be used to identify fluctuations in the signal. At 949 a difference is determined between the candidate local maximum and succeeding data points in the series of sample data that are decreasing from the local maximum. At 950 the difference is compared to a predefined local maximum threshold. At 951 it is determined whether the distance is greater than a maxima threshold. If it is not, the process may return to 949 to check subsequent sample data. If the distance is greater than a maxima threshold, then at 950 the candidate local maximum is classified as a local maximum and candidate local maximum information is written to the FIFO storage. Subsequent data samples may go higher before the next local minimum is found. In such cases, the local maximum is updated based on the higher, subsequent data samples.

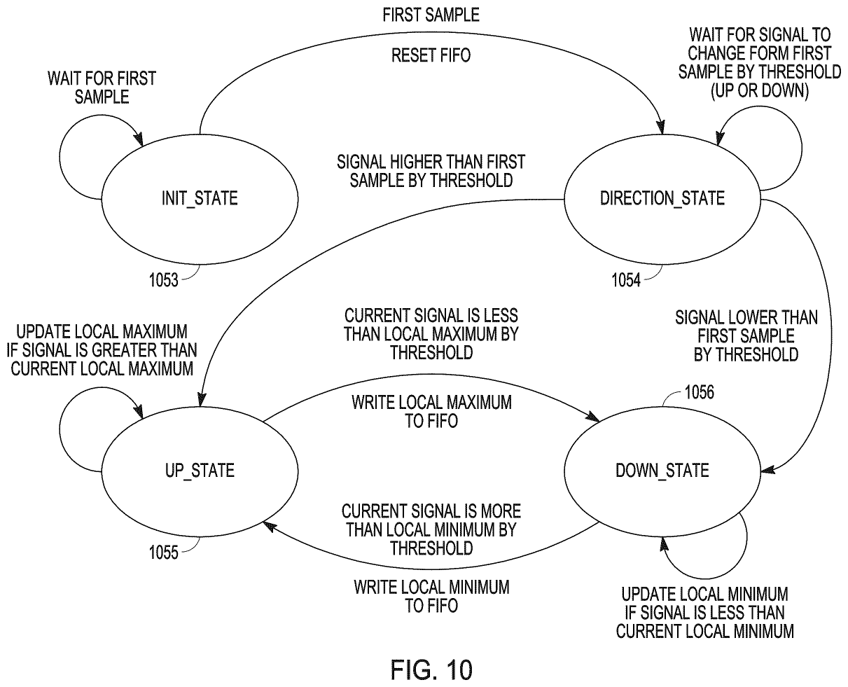

FIG. 10 illustrates, by way of example and not limitation, a state diagram for identifying and capturing local minima and local maxima data in a sensed signal. In the initial state 1053, the system waits for the first data sample. Upon receiving the sample, the system may reset the FIFO storage and enter the direction state 1054 where the system determines the direction of the sample changes. The system may enter the up state 1055 when the subsequent signal samples are higher than the first sample by a threshold or may enter the down state 1056 when the subsequent signal samples are lower than the first sample by a threshold. When in the up state 1055, the system will update the local maximum if a signal sample is greater than the current local maximum. The system will move from the up state 1055 to the down state 1056 and will write the local maximum to FIFO storage when the current signal sample is less than the local maximum by a threshold. When in the down state 1056, the system will update the local minimum if a signal sample is less than the current local minimum. The system will move from the down state 1056 to the up state 1055 and will write the local minimum to FIFO storage when the current signal sample is greater than the local minimum by a threshold. The system can alternate between the up state 1055 and the down state 1056 until the system is reset into the initial state 1053.

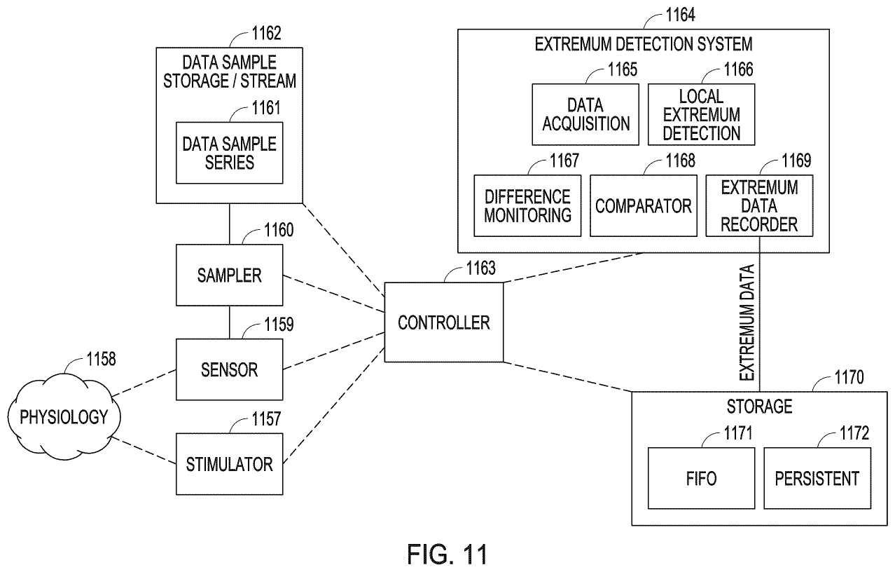

FIG. 11 illustrates, by way of example and not limitation, a system for evaluating data samples from a sensed signal to identify and capture local extrema data. The system may include a stimulator 1157 configured to stimulate a subject, as indicated in the figure by the Physiology cloud 1158. The system may include a sensor 1159 configured to sense a physiological signal from the patient 1158. For example, the stimulator 1157 may deliver a neurostimulation therapy such as, but not limited to, SCS, DBS or PNS therapy. The physiological signal sensed by the sensor 1559 may be used to program the stimulator with stimulation parameters and/or control timing of the neurostimulation. The system may include a sampler 1560 configured to sample the physiological signal sensed by the sensor 1559 to provide a series of data samples 1561 which may be stored in a data sample storage or streamed 1562. The system may include a controller 1563 operationally connected to the stimulator 1157 for programming and/or controlling the stimulator, operationally connected to the sensor 1559 to control the sensor operations, operationally connected to the sampler 1560 to control when and how the signal is sampled, and to the storage or stream 1562 to access the data sample series 1561. The system may include extremum detection system 1564, which may be operationally connected to the controller 1563 and used to detect local maxima and/or local minima in the data sample series. The extremum detection system 1564 may include data acquisition circuitry 1565 configured to access a series of data samples from a sensor signal, local extremum detection circuitry 1566 configured to find a local extremum in the series of data samples, difference monitoring circuitry 1567 configured to determine a difference between the local extremum and the series of data samples subsequent to and receding from the local extremum toward the first direction, comparator circuitry 1568 configured to compare the difference to a predefined threshold, and extremum data recorder circuitry 1569 configured to write extremum data into storage 1570 when the difference exceeds the predefined threshold. The extremum data (e.g., extremum values, chord amplitude, chord length, etc.) may be first written into FIFO storage 1571 and then moved from FIFO storage 1571 to other storage 1572 (e.g., when the FIFO storage is filled or nearly filled). For example, the other storage 1572 may be a persistent storage that provides a processing system with fast access to the data stored therein for analyzing the data and/or operating on the data. The system may operate on the data by controlling the stimulator, adjusting the parameters used by the stimulator and/or providing communication or other alerts to a user, a clinician, a caregiver, and/or a device representative.

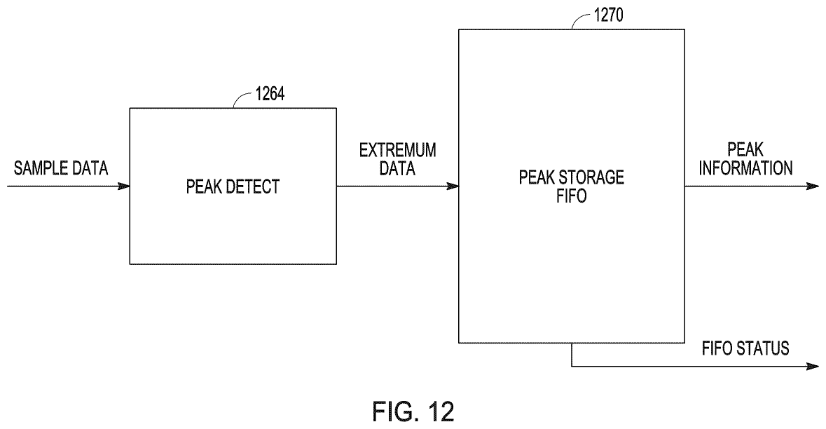

FIG. 12 illustrates, by way of example and not limitation, a system for capturing extrema data from data samples. The illustrated system may generally correspond to the extremum detection system 1164 and storage 1170 in FIG. 12. In the illustrated figure, a peak detection system 1264 may receive sample data and determine when peaks (maximum and/or minimum) is found. Threshold(s) may be used to classify fluctuations as extremum. The peak detection system 1264 may extract extremum data from the peaks and store the extremum data in the FIFO storage 1270. The FIFO storage 1270 may provide the extremum data and may further provide a FIFO status to the system, which may be identify when information should be moved from the FIFO storage to other storage.

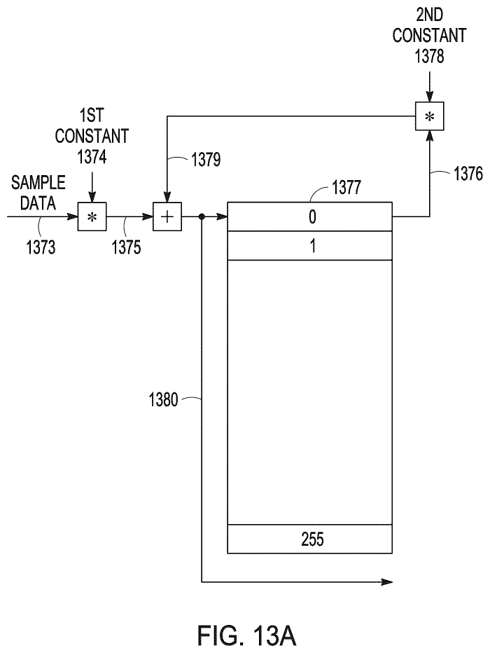

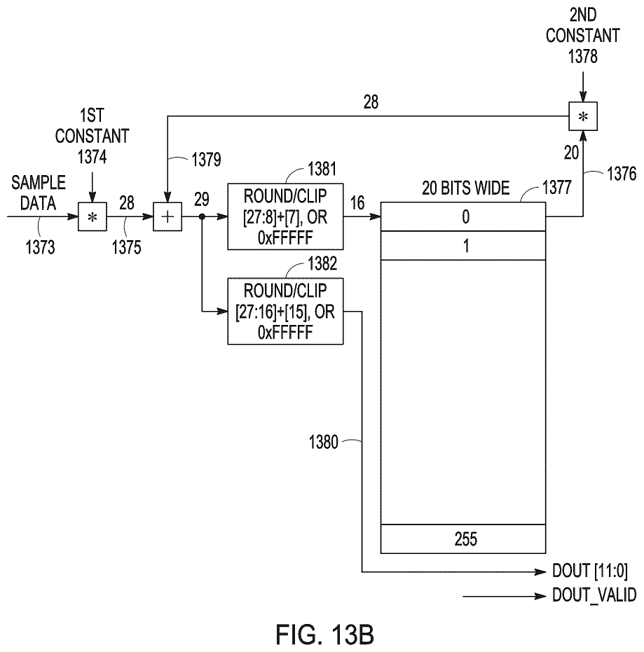

FIGS. 13A and 13B illustrate, by way of example and not limitation, examples of recursive averaging of extrema data. Recursive averaging may be used with relatively predictable signals like ECAPS which are similar. The similar data epochs may be averaged together to further clean up noise. Although recursive averaging is useful for predictable/similar signals, it is not well suited for more random data such local field potentials. Recursive averaging is not likely used with streaming data signals. The recursive averaging helps filter signal noise from the data derived from the signal. The sensed data for a current data epoch 1373 is multiplied by a first constant 1374 to provide a first addend 1375. The filtered data 1376 for a set of previous data epochs (see averaging memory 1377) may be multiplied by a second constant 1378 to provide a second addend 1379. The first and second addends 1375 and 1379 may be summed to tother to provide a value 1380 for the current data epoch. For example, the first constant 1374 for the sampled data may be ⅛ and the second constant 1378 for the feedback data (averaged data epoch from the averaging memory 1377) may be ⅞. FIG. 13B illustrates an example of the recursive averaging in FIG. 13A, but with additional rounding to accommodate differences in the number of bits used in the sampled data and the number of bits stored with each of the previous epochs 1377. For example, the first addend 1375 may have 28 bits, the sum of the first addend 1375 and second addend 1379 may have 29 bits, and each of the stored epochs has 20 bits. The system may include a first rounder 1380 to reduce the number of bits for the first addend from 20 bits to 16 bits, and a second rounder 1381 to reduce the number of bits for the first addend from 20 bits to 12 bits. Recursive averaging may be used with automatic peak detection if the sample window is less than or equal to the size of the averaging memory. The FIFO allows unlimited peaks to be detected as long as the data can be emptied when the FIFO is full. The peak data stored in the FIFO may include peak value, chord length, sample index or distance between sample indexes of the current and previous peak.

Automatic peak detection may work inside of a window of data or with continuous data. The window may be placed at a set point in time after the stimulation pulse that would necessarily include the artifact and evoked potential. The window may be moved back in time to exclude the artifact and/or center the window on the evoked potential. Bounds may be placed on the expected amplitude and time range of the evoked potential to ensure the “found” peak/trough is the desired evoked potential. This may allow sensing to be enabled for a therapy program without needing a user to calibrate the precise position of the sensing window. The sensing window may be sensitive to variations in impedance, lead placement/migration, and other physiological changes. A patient may have many sensing-enabled therapy programs, and some of these programs may not have been run for a relatively long time after the initial programming.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
accessing a series of data samples from a sensor signal;
finding a local extremum in the series of data samples, wherein at least one data sample immediately preceding the local extremum approaches the local extremum from a first direction and at least one data sample immediately succeeding the local extremum recedes from the local extremum toward the first direction;
determining a difference between the local extremum and the series of data samples subsequent to and receding from the local extremum toward the first direction;
comparing the difference to a predefined threshold; and
writing extremum data into storage when the difference exceeds the predefined threshold.

2. The method of claim 1, further comprising using a sensor to sense a biological parameter from a patient and provide the sensor signal, and sampling the sensor signal to provide the series of data samples.

3. The method of claim 2, wherein the series of data samples is accessed from a stream of data samples.

4. The method of claim 2, further comprising delivering electrical stimulation to the patient, and sensing a response to the electrical stimulation by sensing the biological parameter.

5. The method of claim 4, wherein the accessing the sequence of sample data includes accessing sample data within a window after a stimulation pulse and timed to avoid a stimulation artifact and to capture an evoked potential from the delivered electrical stimulation.

6. The method of claim 2, wherein the sensor includes an evoked compound action potential (ECAP) sensor, a local field potential (LFP) sensor, an evoked resonant neural activity (ERNA) sensor, or a cardiac activity sensor.

7. The method of claim 1, wherein the finding the local extremum includes determining a potential extremum using a datapoint in the series of sample data, and updating the potential extremum when the difference did not reach the predefined threshold and a subsequent datapoint in the series of sample data is more extreme.

8. The method of claim 1, wherein when the local extremum is a local maxima, the predefined threshold is a predefined local maxima threshold, the series of sample data points immediately subsequent to the local maxima is less than the local maxima, and local maxima data is written into the storage when the series of sample data points is less than the local maximal by at least the predefined local maxima threshold.

9. The method of claim 1, wherein when the local extremum is a local minima, the predefined threshold is a predefined local minima threshold, the series of sample data points immediately subsequent to the local minima is greater than the local minima, and local minima data is written into the storage when the series of sample data points is more than the local minima by at the predefined local minima threshold.

10. The method of claim 1, wherein the storage includes First In First Out (FIFO) storage, and the writing extremum data into storage includes writing extremum data into the FIFO and then from the FIFO into a persistent memory.

11. The method of claim 1, further comprising performing recursive averaging to reduce noise in finding the local extremum.

12. The method of claim 1, further comprising receiving user-programming via a medical device programmer to set the predefined threshold.

13. The method of claim 1, wherein the extremum data written into the storage includes at least one of:
a value for the extremum data; or
a length or an amplitude of a chord between successive local extremums.

14. The method of claim 1, further comparing the local extremum to an expected extremum to provide a confidence indicator for the extremum date written into the storage.

15. The method of claim 1, wherein the finding, the determining, the comparing, and the writing is performed for each of a plurality of local extremums in the series of sample data points.

16. A non-transitory machine-readable medium including instructions, which when executed by a machine, cause the machine to perform a method comprising:
accessing a series of data samples from a sensor signal;
finding a local extremum in the series of data samples, wherein at least one data sample immediately preceding the local extremum approaches the local extremum from a first direction and at least one data sample immediately succeeding the local extremum recedes from the local extremum toward the first direction;
determining a difference between the local extremum and the series of data samples subsequent to and receding from the local extremum toward the first direction;
comparing the difference to a predefined threshold; and
writing extremum data into storage when the difference exceeds the predefined threshold.

17. A system, comprising:
data acquisition circuitry configured to access a series of data samples from a sensor signal;
local extremum detection circuitry configured to find a local extremum in the series of data samples, wherein at least one data sample immediately preceding the local extremum approaches the local extremum from a first direction and at least one data sample immediately succeeding the local extremum recedes from the local extremum toward the first direction;
difference monitoring circuitry configured to determine a difference between the local extremum and the series of data samples subsequent to and receding from the local extremum toward the first direction;
comparator circuitry configured to compare the difference to a predefined threshold; and
extremum data recorder circuitry configured to write extremum data into storage when the difference exceeds the predefined threshold.

18. The system according to any of claim 17, wherein the local extremum detection circuitry is configured to find the local extremum by determining a potential extremum using a datapoint in the series of sample data, and updating the potential extremum when the difference did not reach the predefined threshold and a subsequent datapoint in the series of sample data is more extreme.

19. The system according to any of claim 17, wherein when the local extremum is a local maxima, the predefined threshold is a predefined local maxima threshold, the series of sample data points immediately subsequent to the local maxima is less than the local maxima, and local maxima data is written into the storage when the series of sample data points is less than the local maximal by at least the predefined local maxima threshold.

20. The system according to any of claim 17, wherein when the local extremum is a local minima, the predefined threshold is a predefined local minima threshold, the series of sample data points immediately subsequent to the local minima is greater than the local minima, and local minima data is written into the storage when the series of sample data points is more than the local minima by at the predefined local minima threshold.
